(19) United States
(12) Patent Application Publication
(10) Pub. No.: US 2025/0312217 A1
(43) Pub. Date: Oct. 9, 2025

(54) PURIFICATION DEVICE

(71) Applicants: NEW COSMOS ELECTRIC CO., LTD., Osaka-shi, Osaka (JP); University of Tsukuba, Tsukuba-shi, Ibaraki (JP)

(72) Inventors: Takeshi Satou, Osaka-shi, Osaka (JP); Makoto Nishii, Osaka-shi, Osaka (JP); Yo Suehiro, Osaka-shi, Osaka (JP); Satoshi Fujiyama, Tsukuba-shi, Ibaraki (JP); Daisuke Hitaka, Tsukuba-shi, Ibaraki (JP); Hirokazu Akahoshi, Tsukuba-shi, Ibaraki (JP)

(21) Appl. No.: 18/865,573

(22) PCT Filed: May 17, 2023
(86) PCT No.: PCT/JP2023/018443

§ 371 Date: Nov. 13, 2024

(30) Foreign Application Priority Data
May 20, 2022 (JP) 2022-083369

(51) Int. Cl. A61G 10/02; A61G 11/00; A61M 16/10; B01D 53/04

(52) U.S. Cl. CPC A61G 10/02; A61G 11/00; A61M 16/105; B01D 53/0446; B01D 2253/102; B01D 2257/708; B01D 2259/4533

(57) ABSTRACT

A purification device for removing volatile substances from air inside an incubator is disclosed. The purification device comprises: an intake part for taking in the air; a suction part for sucking in the air; a removal part for removing volatile substances, which are substances used in medical interventions on an infant patient, from the air sucked in by the suction part; and an exhaust part for exhausting the air from which the volatile substances have been removed by the removal part.

### TECHNICAL FIELD

The present invention relates to a purification device for purifying air by removing volatile substances vaporized in medical interventions.

### BACKGROUND ART

In an incubator, medical procedures are performed on an infant patient being housed therein. For example, in performing the medical procedures on the infant patient in the incubator, volatile substances may be used as alcohols used for the purpose of disinfection or hand sanitization of a medical practitioner, etc., or as solvents for medicines. For example, examples of alcohols used for disinfection and the like include ethanol and isopropanol, etc. If such volatile substances are used in the incubator to vaporize, there is a risk that the infant patient in the incubator would be exposed to the volatile substances for an extended period of time. For example, ethanol and isopropanol may have adverse effects on physical and developmental health of the infant patient. Therefore, when ethanol is used in the medical procedures on the infant patient in the incubator, it is necessary to suppress an increase in concentration of the volatile substances in the air inside the incubator.

### SUMMARY OF THE INVENTION

However, merely removing volatile substances from a space where the incubator is installed, such as inside a hospital room or ward, is not sufficient to remove the volatile substances floating around the infant patient in the incubator.

It is an object of the present disclosure to provide a purification device capable of sufficiently removing volatile substances such as volatile substances floating around an infant patient in an incubator.

The purification device according to one aspect of the present disclosure is a purification device that is arranged in a housing chamber that houses an infant patient in an incubator and purifies air inside the housing chamber, wherein the purification device comprises: an intake part for taking in the air; a suction part for sucking in the air; a removal part for removing volatile substances, which are substances used in medical interventions on the infant patient, from the air sucked in by the suction part; and an exhaust part for exhausting the air from which the volatile substances have been removed by the removal part.

### BRIEF DESCRIPTION OF THE DRAWINGS

FIG. 1 is a schematic perspective view of an incubator equipped with a purification device according to a first embodiment of the present disclosure.

FIG. 2 is a schematic perspective view of an incubator showing another example of the incubator shown in FIG. 1.

FIG. 3 is a schematic plan view of a volatile substance removal device shown in FIG. 1.

FIG. 4 is a cross-sectional view of the volatile substance removal device shown in FIG. 3, taken along A-A line as viewed in the direction of the arrows.

FIG. 5 is a schematic perspective view showing a variation of the purification device according to a second embodiment of the present disclosure.

FIG. 6 is a schematic perspective view showing another variation of the purification device according to the second embodiment of the present disclosure.

FIG. 7 is a schematic cross-sectional view of the purification device shown in FIG. 6.

FIG. 8 is a schematic cross-sectional view of the incubator shown in FIG. 1.

### EMBODIMENT FOR CARRYING OUT THE INVENTION

### First Embodiment

One embodiment of the present invention will be described below in detail. The incubator will be described below taking as an example a closed-type incubator that will be mentioned later.

Some of medical interventions on an infant patient are performed inside the incubator. A purification device that adsorbs and removes volatile substances contained in the air inside the incubator is installed inside the incubator. The volatile substances to be adsorbed and removed by the purification device include substances used in the medical interventions on the infant patient X, such as ethanol.

Thus, the purification device installed inside the incubator removes ethanol, which is a volatile substance used in the medical interventions performed within the incubator, from the air within the incubator. This can improve safety of performing medical interventions on the infant patient within the incubator. It should be noted that one example of the reason for installing the purification device inside the incubator is that it is less likely to cause issues in terms of hygiene and functionality. For example, if all or a part of the purification device is installed outside the incubator, condensation, etc. may occur due to differences in temperature and humidity between the inside and the outside of the incubator, which may easily cause problems in terms of hygiene and functionality. For this reason, the purification device is preferably installed inside the incubator. While ethanol will be described below as an example of volatile substances to be removed, the present invention is not limited to ethanol. First, an overview of the incubator will be described.

### <Overview of Incubator>

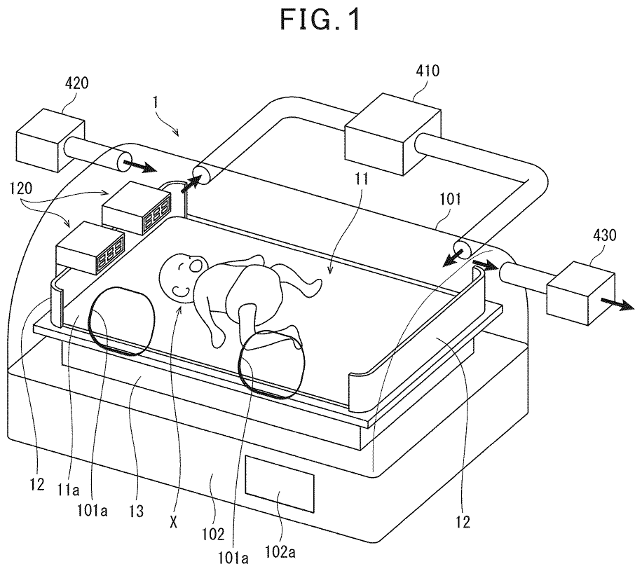

FIG. 1 is a schematic perspective view of an incubator 1. The incubator 1 comprises a housing chamber 101 for housing an infant patient X and a loading stand 102 for loading the housing chamber 101 thereon. Inside the housing chamber 101, a bed 11 and screen parts 12 arranged at the front and the back of the bed are arranged. A surface 11a of the bed 11 is a placing surface on which the infant patient is placed. The bed 11 is installed on a bed stage 13 provided on the loading stand 102. The bed stage 13 is capable of moving the bed 11 in an up-down direction or up-down and left-right directions. The screen part 12 is formed of a transparent resin that has a predetermined height from the surface 11a of the bed 11 and a predetermined width. The screen part 12 is formed of a transparent resin that has a predetermined height from the surface 11a of the bed 11 and a predetermined width, and is also used as an installing part for installing a purification device 120 that will be described later.

The housing chamber 101 is provided with two openings 101a for treatment, allowing a doctor or nurse to perform a treatment such as instillation on the infant patient X. When any treatment is performed on the infant patient X within the housing chamber 101, it is always necessary to perform disinfection. For example, an alcohol swab is used to disinfect a site to be treated such as hands of the infant patient X. If the alcohol swab is used within the housing chamber 101, ethanol is generated within the housing chamber 101. In order to remove the generated ethanol, two purification devices 120, 120 are arranged on the screen part 12 of the bed 11 inside the housing chamber 101.

Here, the air in the housing chamber 101 includes a first air located away from a source that generates ethanol (e.g., alcohol swab) and a second air located near the source that generates ethanol (e.g., alcohol swab). Thus, the purification device 120 primarily removes ethanol from the first air located away from the ethanol source. The purification device 120 is arranged inside the housing chamber 101 and constitutes a removal part for removing ethanol from the air within the housing chamber 101.

The incubator 1 is an incubator (so-called a closed-type incubator) provided with a housing chamber in which internal temperature and humidity can be regulated to house an infant patient. Therefore, the incubator 1 further comprises a circulating part 410, a supplying part 420, and a discharging part 430. The circulating part 410 circulates air, in which the temperature and humidity are regulated, through the housing chamber 101. The supplying part 420 supplies air from the outside to the inside of the housing chamber 101. The discharging part 430 discharges at least part of the air inside the housing chamber 101 to the outside of the housing chamber 101. Thereby, the incubator 1 maintains the temperature and humidity in the housing chamber where the infant patient is housed, and is ventilated by natural intake and exhaust at a level that allows the temperature and humidity to be maintained. It should be noted that the incubator 1 may be capable of adjusting an oxygen concentration in addition to the temperature and humidity.

The loading stand 102 is provided with an operation panel 102a so that a user (doctor, nurse, etc.) can operate the functions, etc. of the incubator 1.

In the incubator 1, the temperature and humidity of the air inside the housing chamber 101 are adjusted by the circulating part 410. Additionally, the purification device 120 takes in air inside the housing chamber 101, removes ethanol contained in the taken-in air, and exhausts the air, from which ethanol has been removed, back into the housing chamber 101. This makes it possible to remove ethanol from the housing chamber 101 while maintaining the temperature and humidity inside the housing chamber 101. Although another method, in which ethanol is removed from the air outside the incubator 1 and the air outside the incubator 1 is replaced with the air inside the incubator 1, is also considered, this method makes it difficult to maintain the temperature and humidity inside the incubator 1.

Furthermore, by arranging the purification device 120 inside the housing chamber 101, it is not required to secure a space for installing the purification device 120 besides the space for installing the incubator 1.

### <Overview of Purification Device>

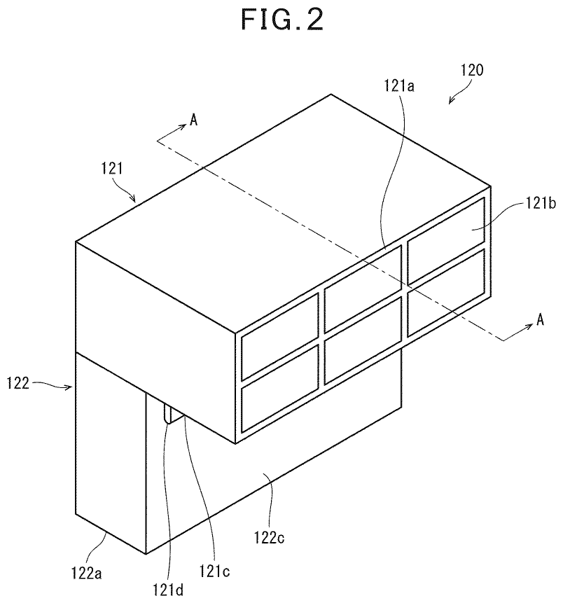

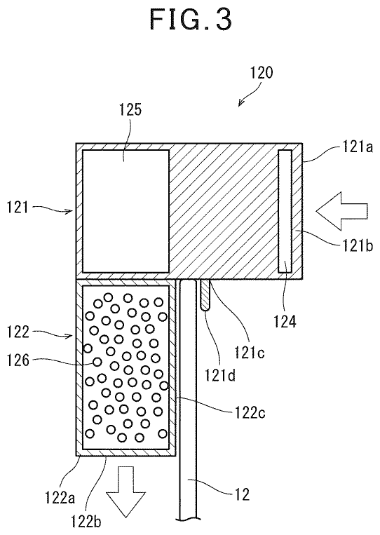

FIG. 2 is a schematic perspective view of a purification device 120. FIG. 3 is a cross-sectional view taken along A-A line as viewed in the direction of the arrows of FIG. 2.

As shown in FIG. 2, the purification device 120 comprises two enclosures (a first enclosure 121 and a second enclosure 122) that are provided integrally. Here, an example is shown in which separate enclosures (a first enclosure 121 and a second enclosure 122) are connected to form a single enclosure. It should be noted that two enclosures (a first enclosure 121 and a second enclosure 122) may be molded integrally into one enclosure. An intake part 121b that takes in outside air is provided on a front surface 121a of the first enclosure 121. The first enclosure 121 is structured to be in communication with the second enclosure 122 at a bottom surface 121c of the first enclosure 121, which is a rear part including a rear surface of the first enclosure 121, and is configured to guide the outside air taken in by the intake part 121b to the second enclosure 122. The rear part of the first enclosure 121 is located on the downstream side of the front surface 121a in a direction from the intake part 121b toward an exhaust part 122b (hereinafter referred to as an exhaust direction). In other words, the front surface 121a is located on the upstream side with respect to the rear part of the first enclosure 121 in the exhaust direction. The front surface of the second enclosure 122 is connected to the bottom surface 121c of the first enclosure 121, which is the rear part including the rear surface of the first enclosure 121. The exhaust part 122b for exhausting the outside air (FIG. 3) is provided on a rear surface 122a of the second enclosure 122 (a surface on a side opposite to the side communicating with the first enclosure 121). The front surface of the second enclosure 122 is located on the upstream side of the rear surface 122a in the exhaust direction. In other words, the rear surface 122a is located on the downstream side of the second enclosure 122 in the exhaust direction.

As shown in FIG. 3, the purification device 120 has an inverted L-shaped cross section and is installed on the screen part 12 inside the housing chamber 101. That is, the purification device 120 is installed above the placing surface. The purification device 120 is provided with fixing parts for fixing the first enclosure 121 and the second enclosure 122 to the inner edge of the housing chamber 101. Specifically, the bottom surface 121c of the first enclosure 121 is provided with a protrusion part 121d as a fixing part, which is parallel to a side surface 122c of the second enclosure 122 and protrudes downward at a position located away from the side surface 122c by a predetermined distance. The protrusion part 121d is a member having a predetermined height and a predetermined width and consisting of, for example, resin, and is provided integrally with the first enclosure 121. FIG. 3 shows an example in which the protrusion part 121d is connected to the first enclosure 121 to form one enclosure. It should be noted that the first enclosure 121 and the protrusion part 121d may be molded integrally into one enclosure.

The protrusion part 121d is provided at a position where a distance between the protrusion part 121d and the side surface 122c of the second enclosure 122 is slightly longer than a thickness of the screen part 12 in the housing chamber 101. Thereby, when the purification device 120 is installed on the screen part 12 provided on the inner edge of the housing chamber 101, the purification device 120 can be stably installed on the screen part 12 by sandwiching the screen part 12 by the side surface 122c of the second enclosure 122 and the protrusion part 121d of the first enclosure 121. In this way, the first enclosure 121 and the second enclosure 122 can be fixed by the protrusion part 121d to the screen part 12 that is a wall part erected on the inner edge of the housing chamber 101. As such, the first enclosure 121 and the second enclosure 122, that is, the main body of the purification device 120, are fixed, by the protrusion part 121d that is the fixing part, to the screen part 12 erected on the inner edge of the housing chamber 101. Thus, the purification device 120 does not interfere with medical procedures on the infant patient X in the incubator 1. It should be noted that the protrusion part 121d may be configured to be detachable from the screen part 12 provided on the inner edge of the housing chamber 101.

In this way, it is preferable to install the purification device 120 at a position that does not interfere with medical procedures. Furthermore, the exhaust direction of the purification device 120 (the direction in which the air, from which ethanol has been removed, is exhausted) is preferably a direction that does not inhibit air circulation inside the incubator 1 and does not come into contact with the infant patient X. Thereby, the incubator 1 maintains its functions (such as the functions of maintaining temperature and humidity), and does not deprive the infant patient X of the body temperature and facilitate a water loss through insensible perspiration upon contact of the air with the infant patient X, so that safety of the infant patient X can be secured.

The protrusion part 121d provided on the first enclosure 121 is provided continuously in a width direction of the first enclosure 121 (a direction perpendicular to the exhaust direction inside the first enclosure 121).

The first enclosure 121 comprises a first removal part 124 and a suction part 125. The first removal part 124 comprises fibrous activated charcoal and quickly adsorbs most of ethanol contained in the outside air taken in from the intake part 121b. Thus, if the amount of ethanol contained in the outside air is small, all of ethanol would be adsorbed in the first removal part 124. In the present embodiment, a sheet-shaped activated charcoal is used, which is obtained by molding a fibrous activated charcoal into a sheet.

The first removal part 124 is of a cartridge type that houses the sheet-shaped activated charcoal in a container that is detachable from the upper side of the first enclosure 121. This makes it easy to replace the first removal part 124. Moreover, the sheet-shaped activated charcoal is disposable. Therefore, the first removal part 124 is replaced as follows. The sheet-shaped activated charcoal housed in the first removal part 124 taken out from the first enclosure 121 is discarded, a new sheet-shaped activated charcoal is housed in the first removal part 124, and the first removal part 124 is mounted to the first enclosure 121 again.

The suction part 125 comprises one sirocco fan, sucks in outside air from the intake part 121b through the first removal part 124, and directs the air to a second removal part 126 at the subsequent stage in a direction perpendicular to a rotation axis direction of the fan. Here, the amount of intake air in the suction part 125 is set to 50 L/min or more, but it is not limited to this value. It should be noted that in order to allow a large amount of air to be taken in, an air intake area of the suction part 125 is preferably large.

The second enclosure 122 comprises a second removal part 126. The second removal part 126 is of a cartridge type that houses pellet-shaped activated charcoal within a cartridge, which is detachable from the second enclosure 122. This makes it possible to easily replace the second removal part 126. Moreover, since the pellet-shaped activated charcoal is disposable, the pellet-shaped activated charcoal housed in the second removal part 126 taken out for replacement is discarded, new pellet-shaped activated charcoal is housed, and the second removal part 126 is mounted to the second enclosure 122 again.

An exhaust direction of air (air from which ethanol has been removed) exhausted from the exhaust part 122b of the purification device 120 having the above-described configuration is set in a direction that does not inhibit air circulation inside the incubator 1.

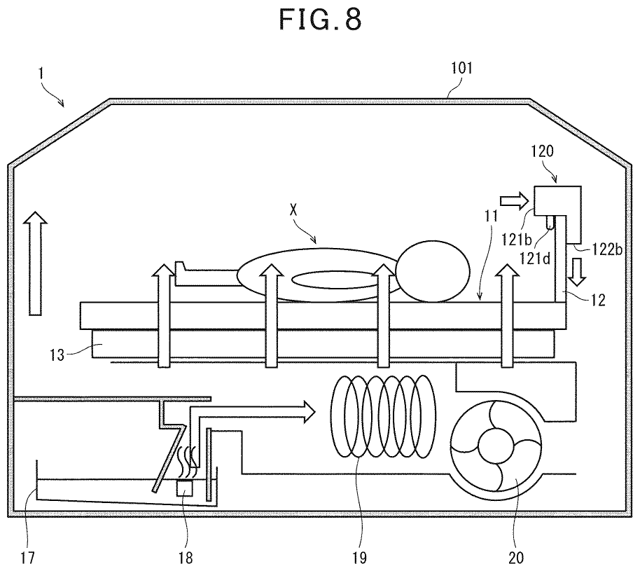

As shown in, for example, FIG. 8, the incubator 1 comprises a water tank 17, a heating part 18, a heater 19, and a fan 20. The water tank 17 houses water. The heating part 18 is provided inside the water tank 17 and generates steam by heating the water housed in the water tank 17. The steam generated by the heating part 18 is circulated inside the housing chamber 101 by the fan 20.

On the other hand, the purification device 120 is fixed to the screen part 12 provided on the bed 11, takes in air inside the housing chamber 101 from the intake part 121b, and exhausts the air from the exhaust part 122b. The air exhausted from the exhaust part 122b is exhausted to the bottom of the bed 11 through a gap between the screen part 12 of the bed 11 and an inner wall surface of the housing chamber 101 of the incubator 1. The air (from which ethanol has been removed) exhausted to the bottom of the bed 11 is sucked in by the fan 20 together with the steam generated by the heating part 18. Thus, the exhaust direction of the purification device 120 coincides with an intake direction in the incubator 1.

Moreover, inside the incubator 1, air circulated inside the incubator 1 is sucked to the bottom of the bed 11 through the gap between the screen part 12 of the bed 11 and the inner wall surface of the housing chamber 101. Thus, if there is a sufficient gap between the screen part 12 of the bed 11 and the inner wall surface of the housing chamber 101, the purification device 120 is preferably installed on the screen part 12 of the bed 11. This allows the exhaust direction of the purification device 220 and the circulating direction of the air circulated inside the incubator 1 to be the same.

As described above, the first removal part 124 in the first enclosure 121, by using the sheet-shaped activated charcoal as an adsorbent, quickly adsorbs a large amount of ethanol with high concentration, and the second removal part 126 in the second enclosure 122, by using the pellet-shaped activated charcoal as an adsorbent, slowly but thoroughly adsorbs any ethanol that has not been completely adsorbed by the first removal part 124.

That is, as shown in FIG. 3, the first removal part 124 is arranged on a side closer to the intake part 121b than the second removal part 126. That is, since the first removal part 124 is arranged on the upstream side of air flow sucked in by the suction part 125 and the second removal part is arranged on the downstream side, the air sucked in from the intake part 121b is removed of ethanol by the first removal part 124 arranged on the side closer to the intake part 121b (upstream side of the air flow) and then removed of ethanol by the second removal part 126 arranged on the downstream side of the air flow, so that any ethanol that has not been completely removed by the first removal part 124 can be removed by the second removal part 126.

One of the conditions for achieving adsorption characteristics of the sheet-shaped activated charcoal used in the above-mentioned first removal part 124 is that it has a large surface area in contact with the air being sucked in.

Thus, the outside air taken in from the intake part 121b of the first enclosure 121 has a large amount of ethanol quickly adsorbed by the first removal part 124, and further, any remaining ethanol is thoroughly adsorbed by the second removal part 126, so that the outside air exhausted from the exhaust part 122b contains almost no ethanol.

The purification device 120 is preferably installed inside the housing chamber 101 of the incubator 1, as shown in FIG. 1. In this way, by installing the purification device 120 inside the housing chamber 101, ethanol can be removed from the air inside the housing chamber 101. This makes it possible to avoid the infant patient X inside the housing chamber 101 of the incubator 1 from being unintentionally exposed to ethanol.

Usually, in the incubator 1, the temperature and humidity of the air inside the housing chamber 101 are adjusted. Thus, as described above, the purification device 120 takes in the air inside the housing chamber 101, guides the air, from which a large amount of ethanol has been quickly removed by the first removal part 124 of the first enclosure 121, to the second removal part 126 in the second enclosure 122, and exhausts the air, from which ethanol has been thoroughly removed by the second removal part 126, into the housing chamber 101. This makes it possible to remove ethanol from the housing chamber 101 while maintaining the temperature and humidity inside the housing chamber 101.

It should be noted that in the first embodiment, the purification device 120 is described, in which the first enclosure 121 comprising the suction part 125 and the second enclosure 122 comprising the second removal part 126 are integrated. In this case, even if efforts are made to slim down each enclosure, it may be difficult to slim down the fan that constitutes the suction part 125 contained in the first enclosure 121, and therefore, it may be difficult to install the purification device 120 on the screen part 12 of the bed 11 inside the housing chamber 101 depending on the incubator 1. Therefore, in a second embodiment below, a purification device is described that can exhaust purified air to the bed 11 even when the space between the screen part 12 of the bed 11 and the housing chamber 101 in the incubator 1 is small.

### Second Embodiment

Another embodiment of the present invention will be described below. It should be noted that for ease of explanation, the same reference numerals are given to members having the same functions as those described in the above-described embodiment, and their explanations are not repeated.

### <Overview of Purification Device>

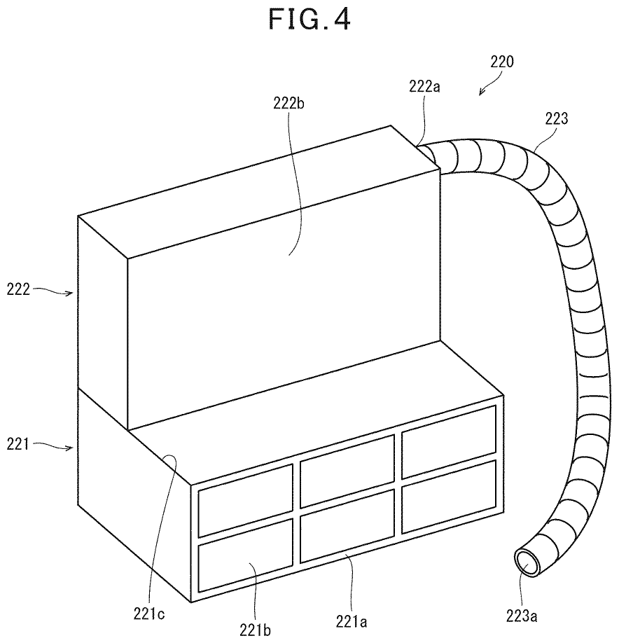

FIG. 4 is a schematic perspective view of a purification device 220 according to this embodiment. The purification device 220 has almost the same structure as the purification device 120 of the first embodiment, except for a structure for exhausting outside air from which ethanol has been removed. Here, correspondence between each element constituting the purification device 220 and each element constituting the purification device 120 of the first embodiment is as follows. A first enclosure 221 corresponds to the first enclosure 121, a second enclosure 222 corresponds to the second enclosure 122, a front surface 221a corresponds to the front surface 121a, an intake part 221b corresponds to the intake part 121b, and an upper surface 221c corresponds to the bottom surface 121c. Furthermore, the first enclosure 221, like the first enclosure 121, comprises a first removal part 124 and a suction part 125 therein, and the second enclosure 222, like the second enclosure 122, comprises a second removal part 126 therein. However, in the second enclosure 222, the outside air after absorbing ethanol is exhausted from a tube 223 provided on a side surface 222a. The tube 223 is a flexible tube having a predetermined length and allows an exhaust port 223a to be directed in a desired direction.

The purification device 220 having the above-described configuration is arranged under the bed 11 in the housing chamber 101 with the first enclosure 221 being on the lower side, and further, the exhaust port 223a of the tube 223 is also arranged under the bed 11. Here, the exhaust port 223a is arranged so that a direction in which the air is exhausted, that is, an exhaust direction, is the same as a direction in which the air circulates inside the housing chamber 101. This allows the purification device 220 to suck in air containing ethanol under the bed 11 and circulate the air, from which ethanol has been removed, under the bed 11 inside the housing chamber 101.

Moreover, since the purification device 220 is used while being placed under the bed 11, it is possible to enlarge the fan in the suction part 125, allowing for improved suction power. In other words, within a range so that the purification device 220 can be placed in a space under the bed 11 inside the housing chamber 101, it is possible to increase the size of the fan in the suction part 125 and improve the suction power. As such, by placing the purification device 220 under the bed 11, it is not required to install the purification device 220 on the screen part 12 of the bed 11. Thus, placing the purification device 220 under the bed 11 is appropriate when the gap between the screen part 12 of the bed 11 and the inner peripheral surface of the housing chamber 101 is not large, i.e., when it is spatially difficult to install the purification device 220 on the screen part 12.

### Third Embodiment

Another embodiment of the present invention will be described below. It should be noted that for ease of explanation, the same reference numerals are given to members having the same functions as those described in the above-described embodiments, and their explanations are not repeated.

### <Overview of Purification Device>

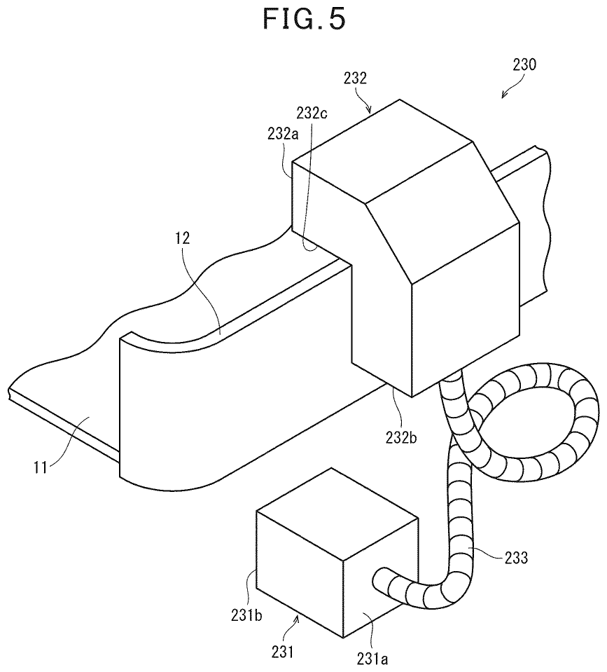

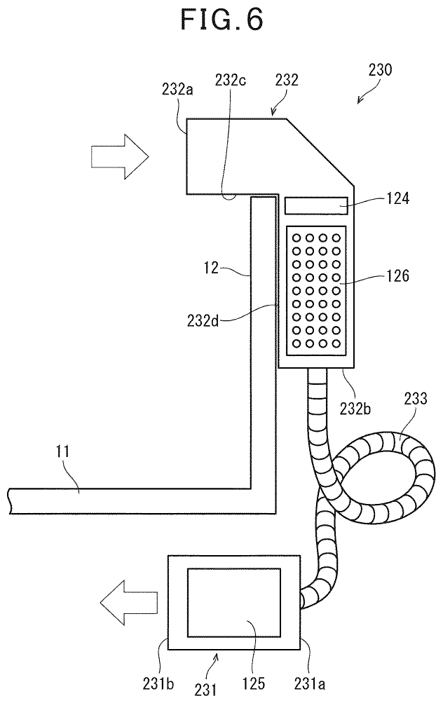

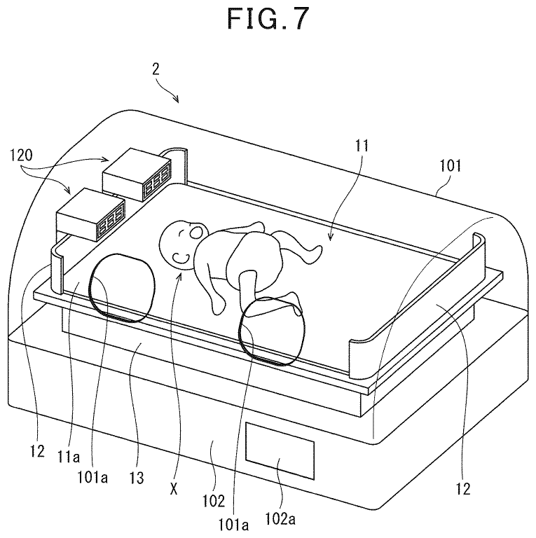

FIG. 5 is a perspective view of a purification device 230 according to this embodiment. FIG. 6 is a schematic cross-sectional view of the purification device shown in FIG. 5. Although, in the above-described second embodiment, the example of the configuration in which the first enclosure 221 and the second enclosure 222 of the purification device 220 are integrated is described, the configuration is not limited to this, and it may be a configuration such as that of the purification device 230 shown in FIGS. 6 and 7. The purification device 230 has a structure in which a first enclosure 231 and a second enclosure 232 are separated and connected by a tube 233. The first enclosure 231 of the purification device 230 corresponds to the first enclosure 221 of the purification device 220, and the second enclosure 232 corresponds to the second enclosure 222.

The first enclosure 231 comprises a suction part 125, which sucks in air from a front surface 231a and exhausts it from a rear surface 231b side. An opening (not shown) that communicates with the inside of the first enclosure 231 is formed on the front surface 231a of the first enclosure 231, and a tube 233 is connected to this opening. The tube 233 is the same as the tube 223 of the purification device 220 shown in FIG. 4.

The second enclosure 232 has a substantially L-shaped cross section and comprises a first removal part 124 and a second removal part 126, which exhaust air, introduced from a front surface 232a, from a rear surface 232b through the first removal part 124 and the second removal part 126. An opening (not shown) that communicates with the inside of the second enclosure 232 is formed on the rear surface 232b of the second enclosure 232, and the tube 233 is connected to this opening. Thus, air exhausted from the second enclosure 232 is sucked in by the first enclosure 231 through the tube 233.

It should be noted that the first removal part 124 adsorbs ethanol using the same sheet-shaped activated charcoal as for the first removal part 124 in the first embodiment, and the second removal part 126 adsorbs ethanol using the same pellet-shaped activated charcoal as for the second removal part 126 in the first embodiment. Moreover, in the first removal part 124 and the second removal part 126, in order to remove ethanol, ethanol may not only be adsorbed using an adsorbent but also be decomposed using a catalyst.

The first enclosure 231 is installed under the bed 11, i.e., below the placing surface, and the second enclosure 232 is installed on the screen part 12 of the bed 11, i.e., above the placing surface. In this case, the second enclosure 232 is installed with a side surface 232d thereof being close to the screen part 12. At this time, a protrusion part, which is the same as the protrusion part 121d described in the first embodiment, is provided on a bottom surface 232c on the upper side of the second enclosure 232, and the second enclosure 232 is fixed to the screen part 12 by this protrusion part. Thus, in the state shown in FIG. 6, the purification device 230 sucks in air containing ethanol from the front surface 232a of the second enclosure 232, and the air, from which ethanol has been removed by the first removal part 124 and the second removal part 126 in the second enclosure 232, is sucked in by the suction part 125 of the first enclosure 231 through the tube 233 and is exhausted from the rear surface 231b of the first enclosure 231 into a space below the bed 11.

According to the purification device 230 having the above-described configuration, the enclosure size of the first enclosure 231 including the suction part 125 is not limited by a size between the screen part 12 and the peripheral wall of the housing chamber 101 of the incubator 1, and the size of the fan can be increased by enlarging the suction part 125, thereby increasing the suction amount. Furthermore, the sizes of first removal part 124 and second removal part 126 in the second enclosure 232 can be increased, making it possible to increase the amount of ethanol adsorbed and increase durability of the adsorbent.

Moreover, a corner of a part bent in the second enclosure 232 (a site facing a part where the bottom surface 232c and the side surface 232d intersect with each other) is chamfered. A range can be thereby widened in which the second enclosure 232 is swung between the screen part 12 and the peripheral wall of the housing chamber 101.

### (Drive Control of Purification Device)

The purification devices 120, 220, and 230 of the first, second and third embodiments are configured to drive and stop the fan in the suction part 125 therein by turning a power source on and off. This power source may be a battery (a primary battery, a rechargeable battery, etc.), an external power source, or a combination of an external power source and a rechargeable battery. When the purification device is installed inside the incubator each time it is used, the battery is preferable as the power source, whereas, when the purification device is installed in the incubator all the time, the external power source is preferable. Usually, the purification device is permanently installed inside the incubator, and when it is operated continuously, the power source is preferably an external power source. Usually, when treatment is performed on the infant patient X inside the housing chamber 101, a practitioner (a doctor or nurse) turns on the power source to drive the fan of the suction part 125. Moreover, a suction power in the purification devices 120, 220, and 230 can be adjusted by a rotation speed of the fan in the suction part 125, so that ethanol can be appropriately sucked in and removed by changing the rotation speed of the fan depending on a concentration of ethanol to be removed.

Furthermore, the purification devices 120, 220, and 230 of the first, second, and third embodiments each have a function of displaying an operating status (a driving status). The function of displaying the operating status refers to explicitly displaying whether the device is operating or not, whether the device is stopped or not, whether a filter (an activated charcoal in a removal part) needs to be replaced or not, whether a trouble occurs or not, whether a battery is lowered or not, etc. In this case, the driving status may be displayed on a display panel or may be displayed by turning on a lamp corresponding to the driving status. This prevents the practitioner from forgetting to operate the purification devices 120, 220, and 230. In addition, the purification devices 120, 220, and 230 can provide a display encouraging replacement of the filter.

Furthermore, it is preferable that the purification devices 120, 220, and 230 of the first, second, and third embodiments are easy to be taken in and out from the opening 101a of the incubator 1 and have a mass for easy handling (e.g., 150 g).

### Variation 1

While in the first embodiment, an example in which the housing chamber 101 is provided with two openings 101a for treatment is described, the number of openings 101a may be three or more as long as it is at least two.

### Variation 2

While in the first embodiment, an example of performing a drive control of the purification device 120 by operation of an operation part provided on each device is described, the drive control may also be performed by operation of the operation panel 102a provided on the incubator 1.

### Variation 3

While in the first embodiment, as shown in FIG. 1, an example in which two purification devices 120 are arranged on one side of the bed 11 (the head side of the infant patient X) is shown, it is not limited to this. Two purification devices 120 may be arranged on the opposite side (the foot side of the infant patient X) or one purification device 120 may be arranged on each side of the bed 11, as long as they do not interfere with medical procedures and ethanol can be appropriately removed. The number of purification devices 120 to be installed may be one, or three or more.

### Variation 4

While in the first embodiment, an example in which the protrusion part 121d is formed integrally with the first enclosure 121 is described, it is not limited to this, and the protrusion part 121d may be provided separately from the first enclosure 121.

### Variation 5

While in the first embodiment, an example in which the protrusion part 121d provided on the first enclosure 121 is provided continuously towards the width direction of the first enclosure 121 (the direction perpendicular to the exhaust direction inside the first enclosure 121) is described, it is not limited to this, and it may be provided intermittently. Moreover, one protrusion part 121d may be provided at each end of the bottom surface 121c of the first enclosure 121 in the width direction (the direction perpendicular to the exhaust direction inside the first enclosure 121). The shape of the protrusion part 121d is not particularly limited, as long as it is a shape that allows the screen part 12 to be sandwiched with the side surface 122c of the second enclosure 122. It should be noted that the protrusion part 121d may be movable so that the distance from the protrusion part 121d to the side surface 122c of the second enclosure 122 is adjusted depending on the thickness of the screen part 12.

### Variation 6

While in the first embodiment, an example of the sheet-shaped activated charcoal is described as the adsorbent used in the first removal part 124, it is not limited to this, and other adsorbents may be used as long as they are capable of adsorbing ethanol.

### Variation 7

While in the first embodiment, an example of using the sirocco fan for blowing out air in the direction perpendicular to the rotation axis direction, as the blowing direction of the outside air flowing from the suction part 125 to the second removal part 126 is perpendicular to the blowing direction of the outside air flowing from the first removal part 124 to the suction part 125, is described, it is not limited to this. The type of the fan may be changed depending on the structures of the first enclosure 121 and the second enclosure 122. For example, if a suction direction and an exhaust direction in the first enclosure 121 are the same, an axial flow fan may be used. Moreover, the number of fans used in the suction part 125 is not limited to two and may be one, or three or more. Thus, the type and number of fans are selected taking into consideration a power supply method, power consumption, a required flow rate, etc., to be adopted for the purification device.

### Variation 8

While in the first embodiment, an example of the pellet-shaped activated charcoal is described as the adsorbent used in the second removal part 126, it is not limited to this, and other adsorbents may be used as long as they are capable of adsorbing ethanol.

### Variation 9

While in the first embodiment, an example in which the adsorbents used in the first removal part 124 and the second removal part 126, respectively, are different from each other is described, they may be the same. Moreover, in order to remove ethanol, ethanol may not only be adsorbed using an adsorbent but also decomposed using a catalyst.

Furthermore, ethanol may be removed both by adsorption and decomposition. For example, each of the first removal part 124 and the second removal part 126 may be provided with both an adsorbent and a catalyst, allowing ethanol to be adsorbed by the adsorbent and decomposed by the catalyst within one removal part. Alternatively, the first removal part 124 may decompose ethanol using a catalyst, and the second removal part 126 may adsorb ethanol using an adsorbent, or the first removal part 124 may adsorb ethanol using an adsorbent, and the second removal part 126 may decompose ethanol using a catalyst.

Moreover, targets to be adsorbed or decomposed in the first removal part 124 and the second removal part 126 may be different. For example, the first removal part 124 may be configured to adsorb or decompose volatile substances other than ethanol, and the second removal part 126 may be configured to adsorb or decompose ethanol, or the first removal part 124 may be configured to adsorb or decompose ethanol, and the second removal part 126 may be configured to adsorb or decompose volatile substances other than ethanol. In addition, the first removal part 124 and the second removal part 126 may be replaceable.

### Variation 10

While in the first embodiment, an example in which one first removal part 124 is provided in the first enclosure 121 and one second removal part 116 is provided in the second enclosure 122 is described, any one of the first removal part 124 and the second removal part 126 may be provided, and two or more removal parts may be provided in each of the first enclosure 121 and the second enclosure 122. Methods of removing volatile substances in these removal parts may be the same or different.

### Variation 11

While in the second embodiment, an example in which the exhaust port 223a of the tube 223 in the purification device 220 is installed at a position under the bed 11 is described, it is not limited to this. The installation position of the exhaust port 223a of the tube 223 in the purification device 220 may be anywhere, as long as it is a position where air can be exhausted to a location that does not affect the infant patient X on the bed 11 and a location that has little effect on circulation inside the incubator 1. In particular, the installation position of the exhaust port 223a of the tube 223 in the purification device 220 may be a position where the air exhausted from the exhaust port 223a is not blown directly onto the infant patient on the bed 11. For example, the exhaust port 223a of the tube 223 may be installed on the bed 11 as long as the air is exhausted toward surrounding walls that constitute the housing chamber 101.

### Variation 12

While in the second embodiment, an example in which the purification device 220 is arranged under the bed 11 is described, the position of the purification device 220 is not limited to this. For example, if there is a sufficient space in the gap between the screen part 12 of the bed 11 and the inner wall surface of the housing chamber 101, the purification device 220 may be installed on the screen part 12 of the bed 11. In this case, the second enclosure 222 is installed with its side surface 222d close to the screen part 12, and the first enclosure 221 positioned on the upper side. At this time, a protrusion part, which is the same as the protrusion part 121d described in the first embodiment, is provided on the upper surface 221c of the first enclosure 221, and the purification device 220 is fixed to the screen part 12 by this protrusion part. In this case, the tube 223 is installed so that the exhaust port 223a of the tube 223 in the purification device 220 faces the space under the bed 11.

### Variation 13

In the components of the purification device 220, it is difficult to reduce the size of the first enclosure 221 comprising the suction part 125, but it is easy to reduce the size, particularly the thickness, of the second enclosure 222, which is a purification part comprising the second removal part 126. Thus, the first enclosure 221 comprising the suction part 125 and the second enclosure 222, which is the purification part, may be configured to be separate, with the first enclosure 221 installed under the bed 11 and the second enclosure 222 installed on the screen part 12 of the bed 11.

Specifically, it is considered that a flexible tube, such as the tube 223 provided in the purification device 220 in FIG. 4, is provided between the first enclosure 221 and the second enclosure 222, and outside air sucked in by the first enclosure 221 is directed to the second enclosure 222 through the flexible tube. In this case, the length of the flexible tube may be set depending on a distance between the installation location of the first enclosure 221 comprising the suction part 125 (under the bed 11) and the installation location of the second enclosure 222, which is the purification part (on the screen part 12).

In the purification device of this variation, by separating the first enclosure 221 comprising the suction part 125 with the fan and the second enclosure 222 comprising the second removal part 126, which is the purification part, it is possible to arrange the first enclosure 221, which is difficult to be reduced in size, under the bed 11, and to install only the second enclosure 222, which is easy to be reduced in size, on the screen part 12. As a result, even if it is difficult to install the first enclosure 221 on the screen part 12 due to space limitations, the air inside the housing chamber 101 can be purified by installing only the second enclosure 222, which constitutes the purification part, on the screen part 12.

### Variation 14

Moreover, the corners of parts bent in the first enclosure 121 in the first embodiment and the second enclosure 222 in the second embodiment may be chamfered. This makes it possible to widen the range in which the first enclosure 121 and the second enclosure 222 can be swung between the screen part 12 and the peripheral wall of the housing chamber 101 when the purification devices 120, 220 are installed in the housing chamber 101.

### Variation 15

While in the purification devices 120, 220, and 230 of the first to third embodiments, drive control is performed by turning the power on and off, it is not limited to this. For example, a concentration sensor for detecting the concentration of ethanol may also be installed inside the housing chamber 101, and the drive control of the purification devices 120, 220, and 230 may be performed based on the detection signals from the concentration sensor. For example, when the concentration of ethanol indicated by the detection signal of the concentration sensor becomes equal to or higher than a predetermined concentration, the fan of the suction part 125 may be initiated, and when the concentration falls below the predetermined concentration, the fan of the suction part 125 may be stopped. Furthermore, if the detection signal of the concentration sensor indicates that the concentration of ethanol has increased further above the predetermined concentration while the purification devices 120, 220, and 230 are operating, the suction power may be increased, such that the rotation speed of the fan in the suction part 125 is increased, to more actively suck in ethanol inside the housing chamber 101. This allows ethanol to be quickly sucked in and removed.

Furthermore, the purification devices 120, 220, and 230 may be provided with detection means for detecting the adsorption state of the first removal part 124 or the second removal part 126. The detection means may be a concentration sensor that detects the concentration of ethanol. In this case, the purification devices 120, 220, and 230 determine the adsorption states of the first removal part 124 and the second removal part 126 (whether they are in a breakthrough state (a state in which adsorption exceeds the adsorption capacity, causing the adsorption target to leak) or whether they are in a sufficient adsorption state) based on the ethanol concentration detected by the detection means.

The detection means may be provided inside the purification devices 120, 220, and 230, or may be provided outside the purification devices 120, 220, and 230. However, the detection means is preferably provided, for example, on the downstream side of the first removal part 124 (between the first removal part 124 and the second removal part 126). This makes it possible to detect the adsorption state of the first removal part 124, allowing the first removal part 124 to be replaced before, for example, it reaches a state where it is no longer able to exhibit a sufficient adsorption function as the adsorption function of the first removal part 124 deteriorates or the second removal part 126 enters a breakthrough state as the amount of adsorption at the second removal part 126 increases. Moreover, detection means may be provided on the downstream side of the second removal part 126 to detect the adsorption state of the second removal part 126. Alternatively, detection means may be provided both between the first removal part 124 and the second removal part 126 and on the downstream side of the second removal part 126 to detect the adsorption states of both the first removal part 124 and the second removal part 126.

Moreover, the drive control of the purification devices 120, 220, and 230 may be performed by combining the detection result from the concentration sensor, which detects a concentration of ethanol and is provided inside the housing chamber 101, with the detection result from the detection means that detects the adsorption state.

Furthermore, detection means may be provided on the upstream side of the first removal part 124. In this case, the detection means detects the concentration of ethanol contained in the air before ethanol is adsorbed by the first removal part 124. That is, the detection means detects the concentration of ethanol contained in the air inside the incubator 1. The drive control of the fan of the suction part 125 may be performed based on the concentration of ethanol detected by the detection means.

In this case, control parts (not shown) of the purification devices 120, 220, and 230 control the motor that rotates the fan of the suction part 125 in accordance with the concentration of ethanol detected by the detection means. For example, if the concentration of ethanol detected by the detection means is higher than a predetermined value, the control part controls the motor so as to increase the rotation speed of the fan, and on the other hand, if the concentration of ethanol detected by the detection means is equal to or lower than the predetermined value, the control part controls the motor so as to decrease the rotation speed of the fan.

Moreover, the purification devices 120, 220, and 230 may comprise a communication part, and the communication part may communicate information indicating the concentration of ethanol detected by the detection means to an external device, thereby monitoring the concentration of ethanol inside the incubator 1. That is, the purification devices 120, 220, and 230 function as monitoring devices that monitor the concentration of ethanol inside the incubator 1.

### Variation 16

In the first embodiment, as shown in FIG. 1, the incubator 1 conducts air circulation inside the incubator 1 through the circulating part 410, air supply into the incubator 1 from the supplying part 420, and air discharge outside the incubator 1 from the discharging part 430, on the upper side of the bed 11 in the housing chamber 101. That is, the main air circulation in the incubator 1 shown in FIG. 1 is conducted on the upper side of the bed 11 in the housing chamber 101. Therefore, there is a possibility that the infant patient X on the bed 11 in the housing chamber 101 may be affected by temperature changes due to air flow. In order to reduce the influence of temperature changes due to air flow on the infant patient X on the bed 11 in the housing chamber 101, it is preferable, for example, in an incubator 2 shown in FIG. 7, to conduct air circulation inside the incubator 2, air supply into the incubator 2, and air discharge outside the incubator 2, on the lower side of the bed 11 in the housing chamber 101. It should be noted that for the discharging direction of air from inside the incubator 2 to outside the incubator 2, it is preferable to discharge the air in a direction that does not inhibit the circulating direction of the air inside the incubator 2 (for example, in the same direction as the circulating direction).

In the incubator 1 shown in FIG. 7, a circulating part 410 and a supplying part 420, which are the same as those in FIG. 1, are arranged at the bottom of the bed 11, although they are not shown in the figure. It should be noted that in the incubator 2 shown in FIG. 7, no device is provided for actively discharging the air in the incubator 2 to the outside, like the discharging part 430 shown in FIG. 1, and instead, the air in the incubator 2 is discharged to the outside through the opening 101a of the housing chamber 101, a small gap between the housing chamber 101 and the placing stand 102, etc.

In the incubator 2 shown in FIG. 7, for the air inside the incubator 2, the air adjusted in temperature and humidity by the circulating part 410 arranged at the bottom of the bed 11 is guided along the inner wall on the long side of the incubator 2 from the bottom of the bed 11 to the top of the housing chamber 101. Moreover, for the air inside the incubator 2, the air above the bed 11 is guided along the inner wall on the short side of the incubator 2 to the circulating part 410 at the bottom of the bed 11. As such, the incubator 2 is configured to reduce the influence of temperature changes on the infant patient X due to air flow.

Thus, according to the incubator 2 shown in FIG. 7, it becomes possible to keep the air inside the incubator 2 clean and to appropriately maintain the temperature and humidity of the air, while reducing the influence of air flow inside the incubator 2 on the infant patient X in the housing chamber 101.

### Variation 17

In order to suppress the influence of the purification device 120 on the infant patient, the intake part 121b of the purification device 120 is present at a position higher than the infant patient. This makes it possible to increase a rate of removal of ethanol and to suppress adverse effects on the infant patient caused by the air flow speed around the infant patient increasing due to intake through the intake part 121b. For example, the intake part 121b of the purification device 120 is located between the top of the screen part 12 of the bed 11 and the fan at the bottom provided within the bed stage 13, and may be set at a height other than the same height as the infant patient. Moreover, the intake part 121b may be installed at a part where the air flow from around the bed 11 reaches a ventilation fan (not shown) inside the incubator 1 (2).

### Variation 18

In the first to third embodiments, the purification devices 120, 220, and 230 may be provided with a sensor for detecting a wind speed (hereinafter referred to as a wind speed sensor) on the intake side. In this case, the control parts in the purification devices 120, 220, and 230 control a motor that rotates fans of suction parts 125, 225 in accordance with a wind speed detected by the wind speed sensor. For example, if the wind speed sensor detects a wind speed within a predetermined range, the control part controls the motor to keep the rotation speeds of the fans of the suction parts 125, 225 constant, if the wind speed sensor detects a wind speed that exceeds the predetermined range and is faster than the predetermined wind speed, the control part controls the motor to reduce the rotation speeds of the fans of the suction parts 125, 225, and if the wind speed sensor detects a wind speed that is slower than the predetermined wind speed, the control part controls the motor to increase the rotation speeds of the fans of the suction part 125, 225. This makes it possible for the purification devices 120, 220, and 230 to prevent the environment inside the incubator 1 (2) from being affected in particular by an excessive actuation of the motor that rotates the fans of the suction parts 125, 225. As such, by providing the fans of the suction parts 125, 225 with a wind speed sensor, it becomes possible to always monitor a wind speed on the intake side. As a result, the fans of the suction parts 125, 225 can make themself suck in an appropriate amount of air.

### Variation 19

The intake parts 121b, 221b of the purification devices 120, 220, and 230 are preferably provided at positions that do not inhibit air circulation in the incubator 1 (2). The intake parts 121b, 221b are particularly preferably provided at positions that do not fluctuate air circulation around the infant patient X inside the incubator 1 (2). Furthermore, the intake parts 121b, 221b are preferably provided between a site where air flows down from the top of the bed 11, in which medical procedures are performed on the infant patient X inside the incubator 1 (2), and a ventilation fan (not shown) of the main body of the incubator 1 (2), so that ethanol is not diffused throughout the whole area of the incubator 1 (2) by an incubator fan (not shown).

### Variation 20

The purification devices 120, 220, and 230 may be provided with a flow rate sensor that detects a flow rate of air exhausted from the exhaust part 122b and the exhaust port 223a. The control parts of the purification devices 120, 220, and 230 determine that the operation of the fan of the suction part 125 is normal if the flow rate detected by the flow sensor is within a predetermined range, and determine that it is abnormal in removal function if the flow rate detected by the flow sensor exceeds the predetermined range. The abnormality in removal function in this case indicates a case where the fan of the suction part 125 is not operating normally or a case where air cannot be exhausted from the exhaust part 122b and the exhaust port 223a. The case where air cannot be exhausted is considered to be a case where the exhaust part 122b and the exhaust port 223a are clogged with a filter clogging or foreign substances. Then, when it is determined that it is abnormal in removal function, the purification devices 120, 220, and 230 notify a practitioner of the abnormality in removal function. This prevents the purification devices 120, 220, and 230 from being continued to be used in a state where ethanol cannot be removed. Accordingly, the purification devices 120, 220, and 230 provided with the flow rate sensors can be said to be preferable devices in terms of safety management. It should be noted that in the purification devices 120, 220, and 230, the flow rate sensors may be provided between the respective first removal part 124 and second removal part 126.

It should be noted that while in each of the above-described embodiments and each of the above-described variations, ethanol is described as an example of volatile substances to be removed, it is not limited to ethanol, and may be another volatile substance such, for example, as isopropanol.

In addition, while in each of the above-described embodiments and each of the above-described variations, a closed-type incubator is described as an example of an incubator, the purification device of the present invention may also be used in an open-type incubator that is not of a closed type.

In the open-type incubator, a housing chamber for housing an infant patient is composed of walls erected around a bed on which the infant patient lies. A purification device may be mounted to these walls to remove volatile substances, such as ethanol, floating in the housing chamber. In addition, the purification device may be suspended from the top of an operating panel (higher than the walls surrounding the bed), on which an operation part is provided in the open-type incubator, to remove volatile substances, such as ethanol, floating in the housing chamber.

A purification device according to one aspect of the present disclosure is a purification device that is installed inside an incubator provided with a housing chamber, in which internal temperature and humidity can be regulated to house an infant patient, and purifies air containing ethanol vaporized due to medical procedures using ethanol on the infant patient housed inside the housing chamber, the purification device comprising: an intake part for taking in the air; a suction part for sucking in the air; a removal part for removing ethanol from the air sucked in by the suction part; an exhaust part for exhausting the air from which ethanol has been removed by the removal part; an enclosure capable of holding at least the removal part; and a fixing part for fixing the enclosure to a part of the housing chamber other than a placing surface on which the infant patient is placed.

According to the above-described configuration, the enclosure constituting the purification device is fixed to a part of the housing chamber other than the placing surface on which the infant patient is placed, so that ethanol in a closed-type incubator can be removed to purify the air without interfering with medical procedures on the infant patient.

An exhaust direction of the air exhausted by the exhaust part may be set in a direction that does not inhibit air circulation inside the closed-type incubator. According to the above-described configuration, ethanol can be removed from the air inside the closed-type incubator without inhibiting air circulation inside the closed-type incubator.

Two removal parts may be held inside the enclosure, and one of the two removal parts may be arranged on a side closer to the intake part than the other removal part. According to the above-described configuration, ethanol is removed from the air, sucked in by the suction part, by one of the removal parts arranged on the side closer to the intake part, and then ethanol is removed by the other removal part, so that any ethanol, which has not been completely removed by the removal part on the side closer to the intake part, can be removed by the other removal part.

The fixing part may be detachably mounted to a wall part erected on an inner edge of the housing chamber. According to the above-described configuration, the fixing part is detachably mounted to the wall part erected on the inner edge of the housing chamber, so that the enclosure can be fixed to the wall part erected on the inner edge of the housing chamber. As such, the fixing part allows the enclosure, i.e., the main body of the purification device, to be fixed to the wall part erected on the inner edge of the housing chamber, so that the purification device does not interfere with medical procedures on the infant patient inside the incubator.

A breakthrough sensor may be provided which detects whether the removal part is in a breakthrough state or not. According to the above-described configuration, the breakthrough sensor can determine whether the removal part is in a breakthrough state or not, so that the removal part can be appropriately replaced.

(1): A purification device according to one aspect of the present disclosure is a purification device that is arranged in a housing chamber that houses an infant patient in an incubator and purifies air inside the housing chamber, the purification device comprising: an intake part for taking in the air; a suction part for sucking in the air; a removal part for removing volatile substances, which are substances used in medical interventions on the infant patient, from the air sucked in by the suction part; and an exhaust part for exhausting the air, from which the volatile substances have been removed by the removal part.

According to the above-described configuration, the removing device comprises a removal part, which is installed in the housing chamber of the incubator and removes predetermined volatile substances from the air in the housing chamber. This allows sufficient removal of volatile substances such as volatile substances floating around the infant patient inside the incubator. Then, this can avoid the infant patient housed in the housing chamber in the incubator from being unintentionally exposed to the volatile substances that are substances used in medical interventions.

(2): The purification device described in (1) may comprise a enclosure capable of holding the removal part and a fixing part for fixing the enclosure to a part of the housing chamber other than a placing surface on which the infant patient is placed.

According to the above-described configuration, the enclosure constituting the purification device is fixed to a part of the housing chamber other than the placing surface on which the infant patient is placed, so that the volatile substances in the housing chamber can be removed to purify the air without interfering with medical procedures on the infant patient.

(3): In the purification device described in (1) or (2), an exhaust direction of the air exhausted by the exhaust part may be set in a direction that does not inhibit air circulation inside the incubator.

According to the above-described configuration, the volatile substances can be removed from the air inside the incubator without inhibiting air circulation inside the incubator.

(4): In the purification device described in any one of (1) to (3), the exhaust direction of the air exhausted by the exhaust part may be set in a direction that does not come into contact with the infant patient.

According to the above-described configuration, it is possible to suppress the infant patient from being deprived of the body temperature and a water loss from being facilitated through insensible perspiration, due to the contact of the air with the infant patient, so that safety of the infant patient can be secured.

(5): In the purification device described in any one of (1) to (4), the incubator may comprise a circulating mechanism for circulating the air inside the incubator, and an exhaust direction of the air exhausted by the exhaust part may coincide with a direction in which the circulating mechanism takes in the air.

According to the above-described configuration, the volatile substances can be removed from the air inside the incubator without inhibiting air circulation inside the incubator.

(6): In the purification device described in any one of (1) to (5), the purification device may be configured so that, when a concentration of the volatile substances in the air inside the housing chamber is higher than a first threshold value, the suction part is driven with a first driving force, and when the concentration of the volatile substances in the air inside the housing chamber is equal to or lower than the first threshold value, the suction part is driven with a driving force lower than the first driving force.

According to the above-described configuration, the volatile substances inside the housing chamber can be efficiently removed while suppressing power consumption.

(7): In the purification device described in any one of (1) to (6), the incubator may be an incubator comprising the housing chamber in which internal temperature and humidity can be regulated to house the infant patient, the incubator may comprise a circulating mechanism for circulating the air inside the incubator, and the removal part may be a removal part for removing the volatile substances from the air circulating inside the incubator.

According to the above-described configuration, the volatile substances can be removed from the air circulating inside the closed-type incubator, and the infant patient can be avoided from being exposed to the volatile substances.

The present invention is not limited to each of the above-mentioned embodiments, and various modifications are possible within the scope of claims. Embodiments obtained by appropriately combining the technical means disclosed in different embodiments, respectively, are also included in the technical scope of the present invention.

### REFERENCE SIGNS LIST

1. Incubator
11. Bed
11a. Surface
12. Screen part
13. Bed stage
101. Housing chamber
101a. Opening
102. Loading stand
102a. Operation panel
120. Purification device
121. First enclosure
121a. Front surface
121b. Intake part
121c. Bottom surface
121d. Protrusion part
122. Second enclosure
122a. Rear surface
122b. Exhaust part
124. First removal part
125. Suction part
126. Second removal part
220. Purification device
221. First enclosure
221a. Front surface
221b. Intake part
222. Second enclosure
222a. Side surface
223. Tube
223a. Exhaust port
X. Infant patient (Treatment subject)

1.-7. (canceled)

8. A purification device that is arranged in a housing chamber that houses an infant patient in an incubator and purifies air inside the housing chamber, the purification device comprising:
an intake part for taking in the air;
a suction part for sucking in the air;
a removal part for removing volatile substances, which are substances used in medical interventions on the infant patient, from the air sucked in by the suction part; and
an exhaust part for exhausting the air from which the volatile substances have been removed by the removal part.

9. The purification device of claim 8, comprising:
an enclosure capable of holding the removal part; and
a fixing part for fixing the enclosure to a part of the housing chamber other than a placing surface on which the infant patient is placed.

10. The purification device of claim 8, wherein an exhaust direction of the air exhausted by the exhaust part is set in a direction that does not inhibit air circulation inside the incubator.

11. The purification device of claim 8, wherein an exhaust direction of the air exhausted by the exhaust part is set in a direction that does not come into contact with the infant patient.

12. The purification device of claim 8,
wherein the incubator comprises a circulating mechanism for circulating the air inside the incubator, and
wherein an exhaust direction of the air exhausted by the exhaust part coincides with a direction in which the circulating mechanism takes in the air.

13. The purification device of claim 8, wherein the purification device is configured so that,
when a concentration of the volatile substances in the air inside the housing chamber is higher than a first threshold value, the suction part is driven with a first driving force, and
when the concentration of the volatile substances in the air inside the housing chamber is equal to or lower than the first threshold value,
the suction part is driven with a driving force lower than the first driving force.

14. The purification device of claim 8,
wherein the incubator is an incubator comprising the housing chamber in which internal temperature and humidity can be regulated to house the infant patient,
wherein the incubator comprises a circulating mechanism for circulating the air inside the incubator, and
wherein the removal part is a removal part for removing the volatile substances from the air circulating inside the incubator.
